(19) United States
(12) Patent Application Publication
(10) Pub. No.: US 2026/0212516 A1
(43) Pub. Date: Jul. 23, 2026

(54) OPHTHALMIC STRUCTURE MODELING BASED EYE REGISTRATION

(71) Applicant: ALCON INC., Fribourg (CH)

(72) Inventor: Simone HUFNAGEL, Berlin (DE)

(21) Appl. No.: 19/444,892

(22) Filed: Jan. 9, 2026

Related U.S. Application Data
(60) Provisional application No. 63/747,398, filed on Jan. 21, 2025.

(51) Int. Cl. G06T 7/33; A61F 9/008; G06T 7/00; G06T 7/13; A61B 17/00

(52) U.S. Cl. CPC G06T 7/337; A61F 9/008; G06T 7/0016; G06T 7/13; A61B 2017/00057; G06T 2207/30041

(57) ABSTRACT

The present disclosure relates to operations that include generating, based on a first image of a first depiction of an eye in a first dilation state and a second image of a second depiction of the eye in a second dilation state, a third depiction of the eye in the second dilation state. Further, the operations may include determining an alignment registration between the first depiction of the eye and the second depiction of the eye based on a comparison between the second depiction and the third depiction.

### CROSS-REFERENCE TO RELATED APPLICATION

This application claims priority to U.S. Provisional Patent Application No. 63/747,398, filed on January 21, 2025, the disclosure of which is incorporated herein by reference in its entirety.

### BACKGROUND

Images of eyes are often used in ophthalmology to diagnose certain eye conditions. Such images may also be used to determine a course of action for treating such conditions. Often ophthalmic surgical systems may be used as part of carrying out a specified treatment plan in which such systems may use the diagnostic images in carrying out such plans. However, the subject eye may be oriented differently during the course of treatment than during the diagnostic stages. As such, the orientation of the subject eye as depicted in the diagnostic image and as used by the ophthalmic surgical system may differ from the orientation of the subject eye during treatment (e.g., the cyclotorsion).

### SUMMARY

The present disclosure relates to operations that include obtaining a first image of a first depiction of an eye in a first dilation state and obtaining a second image of a second depiction of the eye in a second dilation state. In addition, the operations include generating a third depiction of the eye in the second dilation state, the generating of the third depiction including modifying the first depiction based on the second depiction. Moreover, the operations include determining an alignment registration between the first depiction of the eye and the second depiction of the eye based on a comparison between the second depiction and the third depiction.

### BRIEF DESCRIPTION OF THE DRAWINGS

The present disclosure relates to systems and methods for performing registration of an eye based on ophthalmic structure modeling, wherein:

FIG. 1 illustrates an example system configured to perform ophthalmic structure modeling based eye registration, in accordance with one or more embodiments of the present disclosure;

FIG. 2 is a flow diagram illustrating a method for determining an eye registration, in accordance with one or more embodiments of the present disclosure;

FIG. 3A is a flow diagram illustrating a process for generating a third depiction of an eye based on a first depiction of the eye corresponding to a first image and a second depiction of the eye corresponding to a second image, in accordance with one or more embodiments of the present disclosure;

FIG. 3B illustrates an example image of an eye, in accordance with one or more embodiments of the present disclosure;

FIG. 3C illustrates example depictions of an eye, in accordance with one or more embodiments of the present disclosure;

FIG. 3D illustrates an example plot that illustrates relationships between radius positions of portions of an iris structure in resulting depiction modifications as compared to a reference depiction based on different distortion functions, in accordance with one or more embodiments of the present disclosure;

FIG. 4 is a block diagram of an example ophthalmic surgical system, in accordance with one or more embodiments of the present disclosure; and

FIG. 5 is a block diagram of an example computing system suitable for use in implementing one or more embodiments of the present disclosure.

### DETAILED DESCRIPTION

One or more embodiments of the present disclosure relate to registration of an eye between images of the eye. For example, between a diagnostic image taken during planning of a procedure and images at the actual time to receive treatment, there may be misalignment between the eye in the images. Additionally, the eye may be in different dilation states between the images, which may further complicate the ability to align the eye between the images. The present disclosure may provide novel approaches for registration of the eye with respect to the images to correct for misalignment of those images. For example, in some embodiments a non-linear model may be used in adjusting for or predicting varying degrees of changes in the structure of the pupil/iris due to dilation or constriction. As another example, a machine learning system may be used to adjust for or predict such variations in structure due to dilation or constriction. As discussed in detail in the present disclosure, the prediction of structural changes facilitates the eye registration between the images.

In general, reference to “eye registration” in the present disclosure may refer to determining a particular orientation of the eye with respect to a particular reference orientation. In some embodiments, the reference orientation may correspond to the orientation of the eye in a first image of the eye (e.g., a diagnostic image). In these or other embodiments, the registration of the eye may include determining an alignment registration between the first image and a second image of the eye (e.g., an image captured at the time of treatment, or a treatment image).

According to one or more embodiments of the present disclosure, the eye registration may be used and/or performed by one or more ophthalmic surgical systems configured to perform one or more treatment tasks with respect to eyes. For example, in some instances a diagnostic image may be taken of an eye. The diagnostic image may be used to diagnose one or more conditions of the eye and/or to determine a treatment plan for the eye. Additionally or alternatively, the eye may be in a particular state during the capture of the eye for the diagnostic image. For example, in some instances the pupil of the eye may be relatively constricted and/or oriented in a particular manner. In these or other embodiments, the orientation of the eye may be based on how the patient’s head is turned and/or whether the patient’s head is upright or laying down. For example, eyes may rotate within the eye socket—often referred to as cyclotorsion—due to rotations, tilts, positioning, etc. of the head. For example, a typical patient’s eye will rotate around 0-14° when measured from a sitting position versus one in which they are laying down. In the present disclosure, reference to the “orientation” of the eye may be in reference or analogous to the cyclotorsion of the eye.

In some instances, the state of the eye during treatment may be different than the state of the eye in the diagnostic image. For example, the pupil may be significantly more dilated (e.g., through dilating eye drops) or less dilated (e.g., due to brighter illumination during treatment as compared to diagnosis) than during diagnosis. As another example, the pupil may be oriented differently—e.g., the patient’s head may have been upright during diagnosis and may be laying down during treatment. In the present disclosure, reference to the “dilation state of the eye” may be referring to the dilation state of the pupil—e.g., an amount or degree of pupil dilation.

Additionally or alternatively, the ophthalmic surgical system may use the diagnostic image as a reference for performing one or more treatment tasks with respect to the eye. For example, the diagnostic image may identify portions of the eye for which certain treatment procedures may be performed. As such, one or more embodiments of the present disclosure may relate to the ophthalmic surgical system performing eye registration with respect to the diagnostic image (e.g., in which the orientation of the eye in the diagnostic image is used as the reference orientation) such that the ophthalmic surgical system identifies which portions of the eye as currently viewed by the ophthalmic surgical system correspond to the portions for which the procedures are to be performed. In these or other embodiments, the eye registration may include a cyclotorsion registration that may be used to determine or indicate an amount of cyclotorsion between the treatment image and the diagnostic image. Doing so may facilitate alignment of a surgical device at the time of treatment for a treatment task which was planned based on the diagnostic image.

According to one or more embodiments of the present disclosure, the structure of one or more elements of an eye (referred to generally as the “ophthalmic structure” in the present disclosure) may be predicted (or also referred to as “modeled”) for different dilation states of the eye. For example, in some embodiments, the iris structure may be predicted for different dilation states. In these or other embodiments, the modeling may be used to perform registration between different images of the eye.

In particular, in some embodiments a first image of a first depiction of an eye in a first dilation state may be obtained. In these or other embodiments, a second image of a second depiction of the eye in a second dilation state may also be obtained. The first depiction may be modified to generate a third depiction of the eye in the second dilation state such that the ophthalmic structure of the eye in the second dilation state may be modeled from the first depiction. Additionally or alternatively, the modification of the first depiction may be based on the second depiction such that the third depiction may be based on the first depiction and the second depiction. In these or other embodiments, an alignment registration of the eye (e.g., an alignment registration between the first depiction of the eye and the second depiction of the eye) may be determined based on a comparison between the second depiction and the third depiction.

Performing eye registration in the manner described in the present disclosure may help improve the accuracy of the eye registration. For example, many common techniques of eye registration are based mainly on matching features related to the iris. However, such feature matching may be difficult in some instances. For example, in many instances the eye for which a procedure may be performed (also referred to as a “subject eye” in the present disclosure) may be in a relatively constricted state when a corresponding diagnostic image is captured. Further, the pupil of the subject eye may be artificially dilated (e.g., via dilation eyedrops) during the procedure, which may distort many features of the iris and accordingly render iris-based registration difficult or unusable. As a further example, for some treatments the pupil of the subject eye may be more dilated when the corresponding diagnostic image is captured (e.g., due to dark lighting conditions or artificial dilation) while the eye may be in a constricted state (e.g., due to bright illumination) for the treatment. Additionally or alternatively, different illumination or lighting setups that are used with respect to diagnostic images as compared to during treatment may cause different features of the iris to appear differently, which may also cause issues with iris-based registration

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to performance of eye registration in the context of ophthalmic surgical systems. However, such uses are not meant to be limiting such that the eye registration described may be used in any number of different contexts and applications where it may be helpful or applicable.

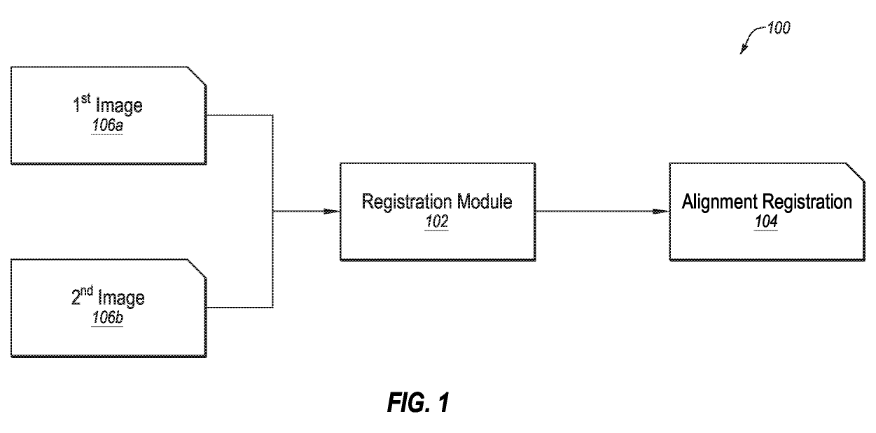

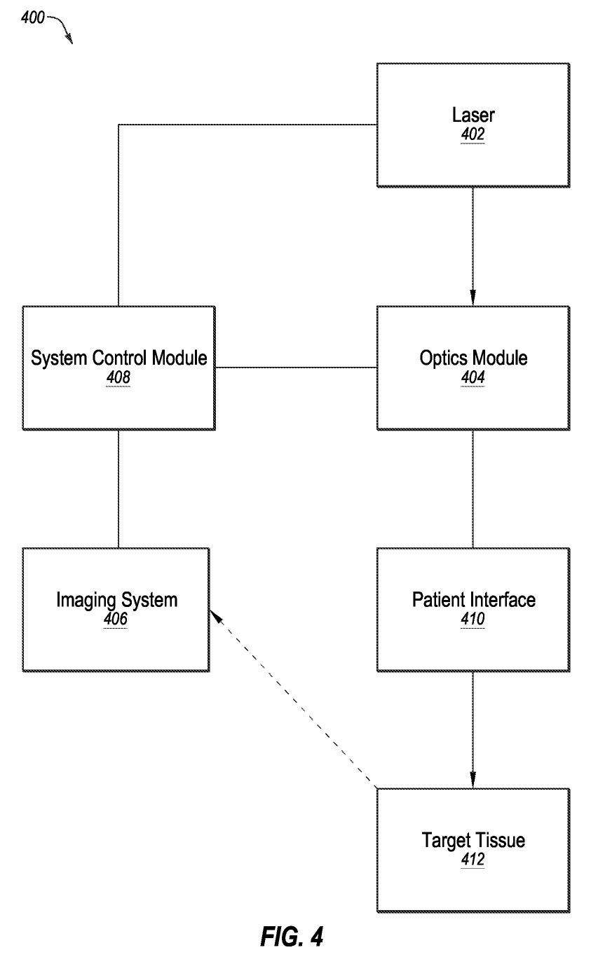

FIG. 1 illustrates an example system 100 configured to perform ophthalmic structure modeling based eye registration, in accordance with one or more embodiments of the present disclosure. In some embodiments, the system 100 may be deployed with respect to an ophthalmic surgical system, such as that described in further detail with respect to FIG. 4 of the present disclosure. The system 100 may include a registration module 102 configured to determine an alignment registration 104 between a first image 106a and a second image 106b.

The first image 106a may be obtained using a first camera which may include any suitable image sensor configured to detect multiple different wavelengths of light—visible and/or non-visible (e.g., infrared)—received through a lens of the first camera. Further, the first camera may be configured to generate the first image 106a based on the detected light in any suitable manner. For example, the first image 106a may include an infrared image of the eye, a visible image of the eye or any suitable combination thereof. In the present disclosure reference to a camera “capturing” an image may include the camera detecting light and generating a corresponding image based on the detected light. Further, in the context of describing the eye as depicted in images, reference to “the eye” or different portions of the eye, such as the pupil, iris, sclera, etc. may refer to the portions of the images that depict the eye, the pupil, the iris, the sclera, etc. and may not necessarily be referring to the eye itself.

Additionally or alternatively, the first image 106a may include a first depiction of the eye that corresponds to a first state of the eye. In some embodiments, the first state may correspond to a first dilation state of the pupil of the eye (also referred to as a dilation state of the eye). For example, in some instances, the first state may include the eye being in a relatively constricted state or in a dilation state (e.g., artificially dilated via dilation eyedrops). In the present disclosure, reference to an eye being “constricted” as compared to being “dilated” may refer to instances in which a difference in pupil dilation satisfies a certain threshold. For example, instances in which the size of the pupil enlarges from a first size to a second size by a threshold percentage (e.g., 50% or more) may be such that a first state corresponding to the first size may be considered “constricted” and a second state corresponding to the second size may be considered “dilated.” For example, a pupil that is referred to as being in a “dilated” state may not actually be fully dilated but may merely be dilated more than in a different state that is referred to as being “constricted.”

Additionally or alternatively, the first state of the eye may include an orientation and/or position of the eye in the first image 106a. In these or other embodiments, the first state of the eye may include an orientation of the eye in the eye socket when a corresponding head is in an upright or laying down position.

In these or other embodiments, the first state of the eye may include a first lighting condition associated with the eye. For example, the first lighting condition may include an ambient light condition of the light around the eye. Additionally or alternatively, the first lighting condition may include an illumination condition with respect to a light that is specifically directed toward the eye. The light that is directed toward the eye may include visible and/or non-visible (e.g., infrared) light. For example, in some embodiments, the eye may be illuminated with an infrared illumination as part of capturing of the first image 106a. Additionally or alternatively, the eye may be illuminated with white light, red light, green light, or any other wavelength of light.

Further, the first image 106a may include first image data associated therewith. The first image data may include information about the first image 106a. For example, the first image 106a may be divided into pixels that are used to create or represent the first image 106a. The pixels may include data associated therewith (referred to as “pixel data”) that describe the features of light that are to be depicted by the pixels. For example, the pixel data may include wavelength information related to the wavelength of detected light corresponding to respective pixels. The wavelength information may indicate a hue or color of visible detected light that corresponds to the respective pixels. Additionally or alternatively, the pixel data may include intensity data that may indicate an intensity (e.g., brightness, magnitude of waveform, etc.) of the detected light corresponding to the respective pixels. In some embodiments the intensity data may specifically correspond to infrared intensity, which may correspond to an infrared illumination in some instances. Additionally or alternatively, the intensity data may correspond to a white light intensity, which may correspond to a white light illumination in some instances. In these or other embodiments, the white light may be broken into different color channels—such as a red channel, a green channel, and a blue channel—and the intensity data may indicate intensities of one or more of the individual channels. Note that in the present disclosure, reference to an image may also refer to corresponding image data associated therewith.

In some embodiments the first image 106a may be a diagnostic image of the eye. The first image 106a may accordingly be used to diagnose one or more conditions (also referred to in the present disclosure as ophthalmic conditions) and/or determine a treatment plan for treatment of the conditions. Additionally or alternatively, the first camera used to capture the first image 106a may be included with any applicable machine or system that may be used for diagnosis of ophthalmic conditions.

The second image 106b may be obtained using a second camera which may include any suitable image sensor configured to detect multiple different wavelengths of light—visible and/or non-visible (e.g., infrared)—received through a lens of the second camera. Further, the second camera may be configured to generate the second image 106b based on the detected light in any suitable manner. In some embodiments, the second camera may be the same camera as the first camera used to capture the first image 106a. Additionally or alternatively, the second camera may be a different camera. In some embodiments, the second image 106b and/or the first image 106a may be one image in a video.

In some embodiments , the second image 106b may include a second depiction of the eye that corresponds to a second state of the eye. In some embodiments, the second state may correspond to a second dilation state of the pupil of the eye. For example, similar to the first state, in some instances, the second state may include the eye being in a relatively constricted state or in a dilation state. Additionally or alternatively, the second state of the eye may include an orientation and/or position of the eye in the second image 106b. In these or other embodiments, the second state of the eye may include an orientation of the eye in the eye socket when a corresponding head is in an upright or laying down position. In these or other embodiments, the second state of the eye may include a second lighting condition associated with the eye that may be selected from options that are analogous to those used for the first lighting condition. Further, the second image 106b may include second image data associated therewith. The second image data may include similar or analogous content to the first image data.

In some embodiments the second image 106b may be associated with performance of a treatment procedure with respect to the eye. For example, in some embodiments, the second camera used to capture the second image 106b may be included in an ophthalmic surgical system configured to perform one or more ophthalmic treatment tasks with respect to the eye. For instance, the ophthalmic surgical system may include a laser configured to perform one or more tasks with respect to the eye, such as a femtosecond laser assist cataract surgery (FLACS) device, a laser assisted in situ keratomileusis (LASIK) surgical device, a small incision lenticule extraction (SMILE) surgical device, or any other laser-based surgical device. The ophthalmic surgical system may use the second image 106b to detect the eye, a location of the eye, etc. Additionally or alternatively, as discussed in further detail in the present disclosure, the second image 106b may also be used to identify the locations of the portions of the eye on which the treatment tasks may be performed.

The registration module 102 may be configured to process the first image 106a and the second image 106b to determine the alignment registration 104. In some embodiments, the registration module 102 may be included with the ophthalmic surgical system that may be used to capture the second image 106b.

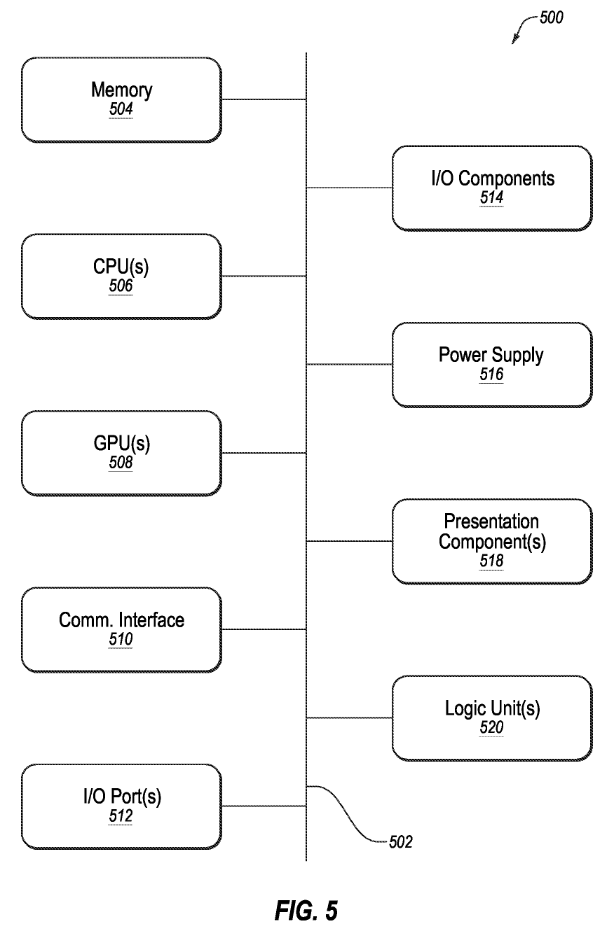

In some embodiments, the registration module 102 may include code and routines configured to allow a computing system to perform one or more registration operations. Additionally or alternatively, the registration module 102 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these or other embodiments, the registration module 102 may be implemented using a combination of hardware and software. In the present disclosure, operations described as being performed by the registration module 102 may include operations that the registration module 102 may direct a corresponding computing system to perform. In these or other embodiments, the registration module 102 may be implemented by one or more computing systems, such as that described in further detail with respect to FIG. 5 of the present disclosure.

In some embodiments, the alignment registration 104 may include a registration between the eye as depicted in the first image 106a and the eye as depicted in the second image 106b. For example, in some embodiments, the alignment registration 104 may include one or more alignment parameters that may indicate the relative orientation and/or position of the eye between the first image 106a and the second image 106b. In these or other embodiments, the alignment parameters may be used to align the eye in the first image 106a with the eye in the second image 106b. For example, the alignment parameters may include one or more rotations, translations, scaling adjustments, etc. applied to one or more of the first image 106a or the second image 106b and that may be used to identify which portions of the first image and the second image depict the same portions of the eye. In these or other embodiments, the alignment registration 104 may accordingly be used to indicate where certain portions of the eye indicated for treatment (also referred to as “treatment portions of the eye”) in the first image 106a may be located in the second image 106b. In the present disclosure, reference of alignment of images may refer to the alignment of the eye as depicted in the images.

Additionally or alternatively, in some embodiments, the alignment registration 104 may include one or more alignment parameters that may be used to determine a registration with respect to the eye itself, such that the alignment registration 104 may include and/or be referred to as an eye registration. For example, in some embodiments, the second image 106b may be captured in real time during performance of an ophthalmic surgical procedure by the ophthalmic surgical system. Further, based on a known location of the second camera, a location of the eye in the second image 106b, and a known relative location of the second camera with respect to a laser beam used to perform treatment tasks, the relative location of the eye itself with respect to the laser beam may be determined. In addition, as indicated above, the alignment registration 104 may be used to identify the locations of certain portions of the eye in the second image 106b (e.g., treatment portions of the eye), which may then be used to determine the relative locations of the portions with respect to the laser beam.

In these or other embodiments, the alignment registration 104 may include a cyclotorsion registration with respect to the eye. For example, the first state of the eye with respect to the first image 106a may be based on the head of the patient being upright during capture of the first image 106a. Further, the second state of the eye with respect to the second image 106b may be based on the head of the patient laying down during capture of the second image 106b. As such, the eye may be rotated to some degree in the second image 106b as compared to the first image 106a. The alignment registration 104 may indicate the degree of such cyclotorsion between the first image 106a and the second image 106b as a cyclotorsion registration. In some embodiments, the cyclotorsion registration may be indicated by a radial measurement (e.g., degrees, radians) that may indicate the amount of rotation.

In some embodiments and as indicated above, the registration module 102 may be configured to determine the alignment registration 104 by modeling ophthalmic structure changes (e.g., iris structure changes) that may occur between different dilation states (e.g., between the first dilation state and the second dilation state). For example, in some embodiments, the alignment registration may be based on a third depiction of the eye that is a modified version of the first depiction of the eye as included in the first image 106a in which the third depiction depicts the eye in the second dilation state.

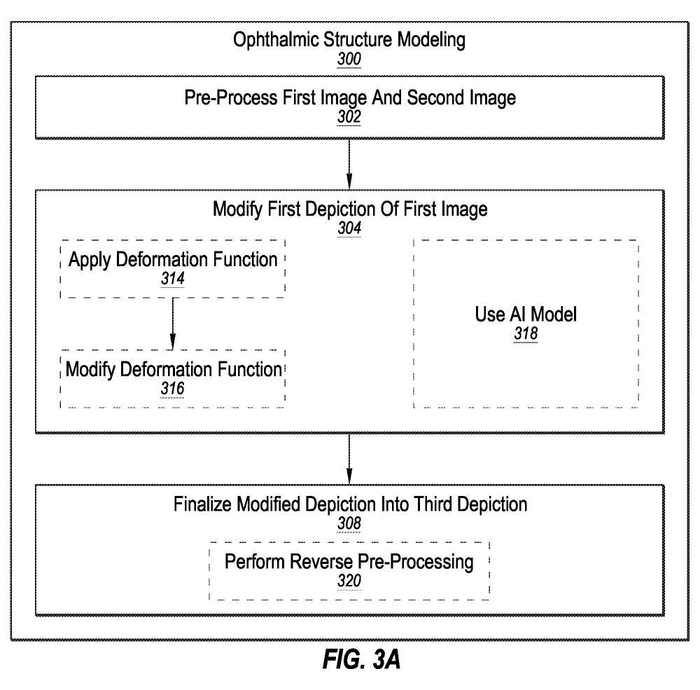

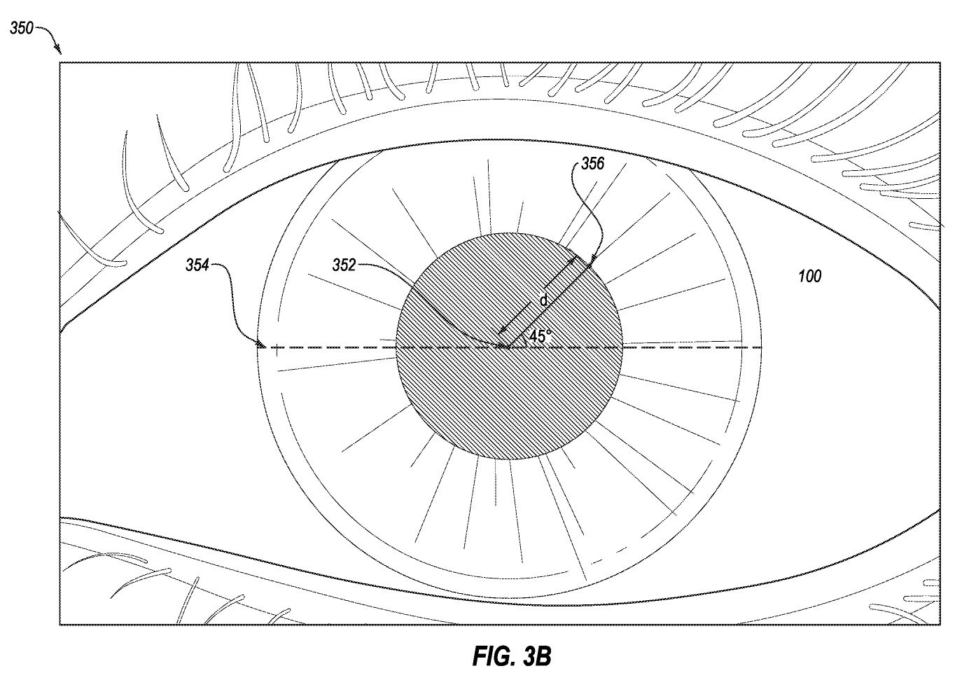

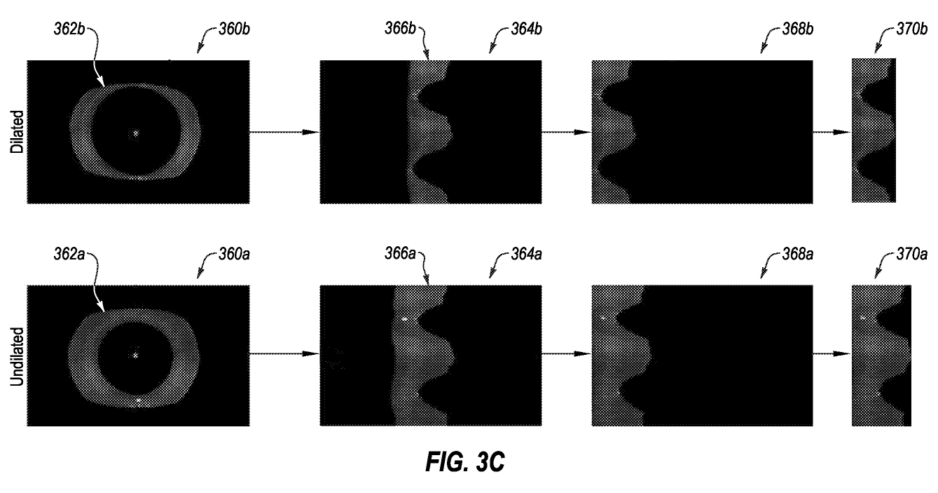

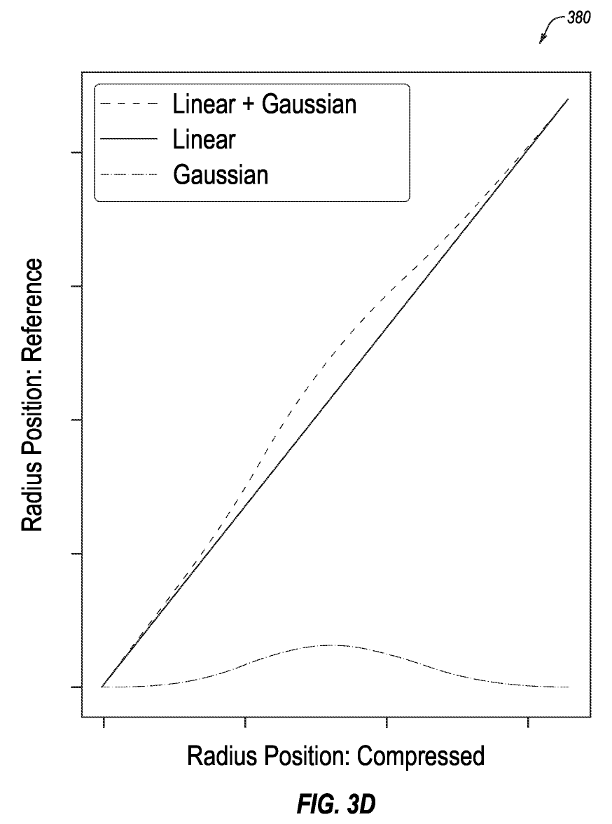

The third depiction may be determined by predicting the ophthalmic structure changes that may occur from the eye transitioning from the first dilation state to the second dilation state and then applying such predictions to the first depiction to obtain the third depiction. For example, in some embodiments, a deformation function may be applied to the first depiction to obtain a modified depiction of the eye as compared to the first depiction in which the third depiction may be based on (or may be) the modified depiction. The deformation function may be configured to adjust the dilation state of the eye at respective radial positions of the iris based on a difference between the first dilation state and the second dilation state. In these or other embodiments, the deformation function may be modified, adjusted, or updated based on a comparison between the second depiction and the modified depiction such that the resulting third depiction may more closely match the second depiction with respect to ophthalmic structure. In some embodiments, one or more operations described with respect to FIGS. 3A-3D may be performed to obtain the third depiction using the deformation function.

Additionally or alternatively, the third depiction may be determined through the use of an artificial intelligence (AI) model included in the registration module 102. The AI model may include any suitable model that may be trained to modify the depiction of an eye from a first particular dilation state to a second particular dilation state. For example, the AI model may include a neural network, a machine learning model, a deep learning neural network, a generative language model (e.g., a large language model (LLM), vision language model (VLM), multi-modal language model (MMLM), and/or any other applicable type of generative AI). In some embodiments, one or more operations described with respect to FIG. 3A may be performed to obtain the third depiction using an AI model.

In these or other embodiments, the registration module 102 may be configured to determine the alignment registration 104 based on a comparison between the third depiction and the second depiction. For example, the orientation of the first depiction may be maintained for the third depiction such that the cyclotorsion of the eye in the third depiction may differ from that of the second depiction. Further, the overall shape of the iris and/or different portions of the iris (e.g., the edge of the iris) in the second depiction and the third depiction may be irregular. However, the shape of the iris and/or its corresponding irregularities in the second depiction and in the third depiction may be relatively the same or similar but may not be aligned—e.g., due to differences in the cyclotorsion. In these or other embodiments, the registration module 102 may be configured to align the overall shape of the iris and/or one or more irregularities of the iris between the third depiction and the second depiction. The changes in angle, orientation, position, etc. to align the third depiction with the second depiction may be used to identify the alignment parameters that may be included in the alignment registration 104.

Modifications, additions, or omissions may be made to FIG. 1 without departing from the scope of the present disclosure. For example, the system 100 may include more or fewer elements depending on the implementation. For instance, the registration module 102 may be configured to determine multiple different alignment registrations 104 based on multiple comparisons between the first image 106a and multiple other second images. Additionally or alternatively, although described as being deployed with respect to an ophthalmic surgical system, in some embodiments, the registration module 102 may be deployed with respect to any suitable application where registration of an eye may be applicable. In addition, although the above description relates to generating the third depiction by modifying the first depiction and then performing the registration between the third depiction and the second depiction other embodiments may include generating the third depiction by modifying the second depiction and then performing the registration between the third depiction and the first depiction.

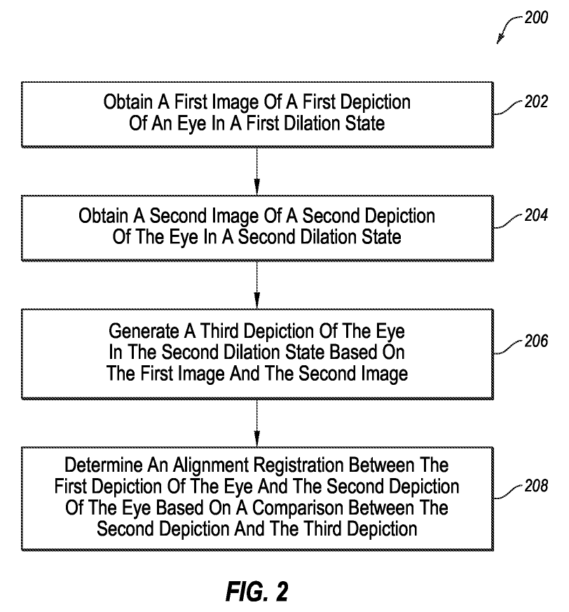

FIG. 2 is a flow diagram illustrating a method 200 for determining an alignment registration, in accordance with one or more embodiments of the present disclosure. One or more operations of the method 200 may be performed by any suitable system, apparatus, or device such as, for example, the system 100 of FIG. 1, an ophthalmic surgical system such as that described with respect to FIG. 4 of the present disclosure, and/or a computing system such as that described with respect to FIG. 5 of the present disclosure.

The method 200 may include a block 202. Block 202 may include obtaining a first image of a first depiction of an eye in a first dilation state. For example, the first image 106a of FIG. 1 may be an example of the obtained first image.

In these or other embodiments, the method 200 may include a block 204. Block 204 may include obtaining a second image of a second depiction of an eye in a second dilation state. For example, the second image 106b of FIG. 1 may be an example of the obtained second image.

Additionally or alternatively, the method 200 may include a block 206. Block 206 may include generating a third depiction of the eye in the second dilation state based on the first image and the second image. In some embodiments, the third depiction of the eye may be generated by modeling changes in an ophthalmic structure of the eye that may occur in a transition between the first dilation state and the second dilation state. In these or other embodiments, the generating of the third depiction may include one or more operations described with respect to FIGS. 3A-3D of the present disclosure.

In these or other embodiments, the method 200 may include a block 208. Block 208 may include determining an alignment registration between the first depiction of the eye and the second depiction of the eye based on a comparison between the second depiction and the third depiction. For example, the specific shape and/or size of a first transition edge between the outer edge of the pupil and the inner edge of the iris may not be uniform such that the first transition edge may include one or more first unique characteristics. Additionally or alternatively, the specific shape and/or size of a second transition edge between the outer edge of the iris and the inner edge of the sclera may also not be uniform such that the second transition edge may include one or more second unique characteristics.

In some embodiments, the alignment registration may include comparing the relative locations of one or more first unique characteristics and/or second unique characteristics in the respective transition edges of the third depiction and the second depiction. In these or other embodiments, the differences in the relative locations may be used to determine one or more of the alignment parameters of the alignment registration.

For example, given that the third depiction and the second depiction may correspond to a same dilation state, the differences in the relative locations may correspond to cyclotorsion differences. As such, the cyclotorsion angle between the third depiction and the second depiction may be determined. Further, the eye may be oriented in the third depiction in the same manner that the eye is oriented in the first depiction such that the cyclotorsion angle between the third depiction and the second depiction may be the same as or substantially the same as the cyclotorsion angle between the first depiction and the second depiction. As such, the cyclotorsion angle determined with respect to the third depiction and the second depiction may be included in the alignment parameters of the alignment registration between the first depiction and the second depiction.

Additionally or alternatively, in some embodiments, the alignment registration may include applying any suitable optimization functions or techniques to align the third depiction and the second depiction based on the unique characteristics of the transition edges. In these or other embodiments, one or more of the alignment parameters may be determined based on the resulting optimization determination and based on the third depiction being oriented the same as the first depiction such as described in the preceding paragraph.

Modifications, additions, or omissions may be made to the method 200 without departing from the scope of the present disclosure. For example, the operations of method 200 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

For example, although the above description relates to generating the third depiction by modifying the first depiction and then performing the registration between the third depiction and the second depiction other embodiments may include generating the third depiction by modifying the second depiction and then performing the registration between the third depiction and the first depiction. In addition, in some instances the third depiction may correspond to a dilated state and in other instances the third depiction may correspond to a constricted or undilated state.

Additionally or alternatively, in some embodiments the method 200 may include one or more adjustment operations related to an ophthalmic surgical system. For example, in some embodiments, an orientation of the eye with respect to the ophthalmic surgical system may be included in or determined based on the alignment parameters. For instance, an orientation of the eye with respect to one or more laser beams generated by one or more lasers of the ophthalmic surgical system may be determined based on or included in the alignment parameters and based on a known positional relationship between the camera used to capture the second image and the laser beams. Such a determination may be performed using any suitable technique.

In these or other embodiments, the one or more adjustment operations may include adjusting a position of the ophthalmic surgical system (e.g., adjusting positioning of the laser beams) based on the alignment parameters. For example, a particular portion of the eye may be designated for treatment (e.g., based on the first image) and the alignment parameters may be used to orient laser beams with respect to such portion to allow corresponding laser-based treatment tasks to be performed with respect to the particular portion. In some embodiments, the adjusting of the orientation may include physically moving the positions of the laser beams—e.g., via one or more actuators or motors that move the laser itself and/or by adjusting one or more laser parameters that adjust the location of the laser beam without moving the whole laser (e.g., via adjustment of mirrors, etc.). Additionally or alternatively, the adjusting of the orientation may include changing the pattern in which the laser is programmed to treat the eye such that the treatment pattern is rotated in software settings of the laser to adjust the pattern in which the laser will be fired. Additionally or alternatively, the adjusting of the orientation may include changing a pattern of the laser beams. In these or other embodiments, the adjustment of the orientation may include adjusting the position of the eye (e.g., by adjusting an orientation of a bed on which the patient is laying). In the present disclosure, reference to a “laser beam” may include any suitable beam of light that may be generated by a laser. Such light beams may be continuous or pulsed.

FIG. 3A is a flow diagram illustrating a process 300 for generating a third depiction of an eye based on a first depiction of the eye corresponding to a first image and a second depiction of the eye corresponding to a second image, in accordance with one or more embodiments of the present disclosure. One or more operations of the process 300 may be performed by any suitable system, apparatus, or device such as, for example, the system 100 of FIG. 1, an ophthalmic surgical system such as that described with respect to FIG. 4 of the present disclosure, and/or a computing system such as that described with respect to FIG. 5 of the present disclosure. Additionally or alternatively, as mentioned in the present disclosure, one or more of the operations of the process 300 may be used and/or included in the block 206 of the method 200 of FIG. 2.

In general, the modeling of the process 300 may be used to model ophthalmic structure changes (e.g., iris structure changes) that may occur between dilation states of an eye. In these or other embodiments, the modeling may be based on a first image of a first depiction of an eye in a first dilation state and a second image of a second depiction of the eye in a second dilation state—such as the first image 106a and the second image 106b, respectively, of FIG. 1. Further, in the present description, the modeling may be such that the first depiction may be modified into a third depiction having the second dilation state. However, the process 300 may also be performed to modify the second depiction into a third depiction having the first dilation state without departing from the scope of the present disclosure.

In some embodiments, the process 300 may include a pre-processing block 302. The pre-processing block may include one or more pre-processing operations that may be performed on the first image and/or the second image (referred to generically and generally as “the images”).

In some embodiments, the pre-processing operations may include converting the images to a polar coordinate system. For example, in some embodiments, the center of the pupil may be identified in the images using any suitable technique. Additionally or alternatively, the center of the pupil may be used as the origin for the polar coordinate system and a horizontal line running through the origin may be used as the x-axis. In these or other embodiments, individual locations or points within the image (e.g., pixels) are indicated with a radial coordinate “r” indicating a distance of the respective locations from the origin (e.g., center of the pupil) and an angular coordinate corresponding to the angle between the x-axis and a line segment between the origin and respective locations, which may also be referred to as an “angular location” in the present disclosure.

For example, FIG. 3B illustrates an example image 350 of an eye. A center 352 of the pupil may be used as the origin and a line 354 may be used as the x-axis for the polar coordinate system. For instance, a point 356, located a distance “d” from the center 352 and located along a line segment that has an angular location 45° offset from the line 354 may have an “r” coordinate of “d” and an angular coordinate of “θ = 45°”, which may be expressed as “(d, θ)”.

Returning to FIG. 3A, in these or other embodiments, the positions of one or more ophthalmic structures may accordingly be represented using the polar coordinates. For example, the position of the iris may be indicated with the polar coordinates that are associated with the iris. In the present disclosure, the structure of the iris values in the polar coordinate system may be referred to as the “iris band.”

In some embodiments, the depictions of the eye may be “unrolled” based on the polar coordinates. For example, FIG. 3C illustrates an example first depiction 360a of the eye in an undilated (also referred to as “compressed”) state and an example second depiction 360b of the eye in a dilation state prior to unrolling. In addition, the first depiction 360a includes a first iris portion 362a that is lighter than the rest of the eye in the first depiction 360a and that depicts the iris. Further, the second depiction 360b includes a second iris portion 362b that is lighter than the rest of the eye in the second depiction 360b and that depicts the iris. As illustrated between the first iris portion 362a and the second iris portion 362b, the iris may be compressed in the second depiction 360b as compared to the first depiction 360a due to the differences in dilation states.

Further, FIG. 3C illustrates an example unrolled first depiction 364a of the first depiction 360a and an unrolled second depiction 364b of the second depiction 360b (generally referred to as “unrolled depictions 364”). In the unrolled depictions 364, the x-axis corresponds to the radial coordinates in the polar coordinate system and the y-axis corresponds to the angular coordinates. Further, the first and second depictions 364a and 364b may include first and second iris bands 366a and 366b, respectively, of the iris that may respectively correspond to first and second iris portions 362a and 362b.

In some embodiments, the pre-processing may further include adjusting the radial positions within the transformed images. For example, the boundary between the pupil and the iris may not necessarily have the same radial coordinate at each angle due to the pupil not necessarily being perfectly circular. To correct for such irregularities, the radial position of the border between the pupil and the iris may be aligned with respect to each angular position to generate shifted depictions—e.g., via shifting the radial position for the border at each angular position to be zero. For example, FIG. 3C illustrates first and second shifted depictions 368a and 368b, respectively, that shift the radial positions of the border between the pupil and the first and second iris bands 366a and 366b to zero.

In these or other embodiments, the intensity values at the border for each angular position may also be adjusted as part of the alignment. For example, in some embodiments, the adjustment may include calculating the position of the interface between the pupil and iris for each row in the image (e.g., for each angular position). For each row, the content of the row may be shifted—e.g., by changing the image intensities—such that this interface position is at column “0.”

In some embodiments, the pre-processing may include cropping the shifted depictions to generate cropped depictions. For example, in some embodiments, the shifted depictions may be cropped to mainly include the iris bands. In these or other embodiments, the cropping may include cropping at least a portion of the edge of the iris band between the pupil and the iris band such that the resulting edge is uniform rather than potentially having differences due to the irregularities in the pupil shape. FIG. 3C illustrates example first and second cropped depictions 370a and 370b that are cropped versions of the first and second shifted depictions 368a and 368b, respectively. For example, as illustrated in the first and second cropped depictions 370a and 370b, the image may be cropped to the radial position in which the first iris image pixels are detected or observed. Additionally or alternatively, a small number of pixels (e.g., 1, 2, 5, 10) may be maintained beyond the first iris image pixels.

In some embodiments, certain preprocessing steps may be omitted. For example, rather than aligning the interface at zero as illustrated in the first and second shifted depictions 368a and 368b, the original alignment may be maintained and the image may be cropped to the radial position where the first iris image pixels are detected. Additionally or alternatively, a small number of pixels (e.g., 1, 2, 5, 10) may be maintained prior to the first iris image pixels.

Returning to FIG. 3A, in some embodiments, the process 300 may also include a block 304 at which the first depiction of the first image may be modified to generate a modified depiction. In the present disclosure, reference to the modifying the first depiction in the context of

block 304 may include modifying the first depiction without any pre-processing having been performed with respect to the first depiction and/or modifying one or more altered versions of the first depiction after one or more pre-processing operations have been thereto performed. For example, referring to FIG. 3C, reference to modifying the first depiction 360a in the context of block 304 may include modifying the first unrolled depiction 364a, the first shifted depiction 368a, and/or the first cropped depiction 370a.

In general, the modifying of the first depiction may include adjusting the first depiction from the first dilation state to the second dilation state. In some embodiments, the amount of adjustment used may be based on a comparison between the first depiction and the second depiction. For example, a difference in relative radial positions of one or more of the transition edges of the iris (e.g., the border between the pupil and the iris and the border between the iris and the sclera) between the first depiction and the second depiction may be determined. Such a difference may indicate the difference between the first dilation state and the second dilation state and may indicate the amount of dilation adjustment that may be made to the first depiction to transition from the first dilation state to the second dilation state.

In these or other embodiments, the adjusting may include adjusting one or more ophthalmic structure elements based on the constriction or stretching that may occur to such ophthalmic structures due to changes from the first dilation state to the second dilation state. For example, the first dilation state may be a constricted (or undilated) state and the second dilation state may be a dilated state. In such an instance, the adjusting may include modeling compression of the iris structure that may occur in instances in which the pupil transitions from a constricted to a dilated state. Similarly, in instances in which the first dilation state is a dilated state and the second dilation state is a constricted state, the adjusting may include modeling expansion of the iris structure that may occur in instances in which the pupil transitions from a dilated to a constricted state.

In some embodiments, the modifying of the first depiction may include a block 314 that includes applying a deformation function to the first depiction. The deformation function may model the deformation of the iris structure that may occur with respect to the transition from the first dilation state to the second dilation state. In some embodiments, the deformation function may differ from more traditional functions used to model the deformation of the iris between different dilation states. For example, some traditional functions may assume a linear deformation in which the radial positions of the iris at each angular location are uniformly and linearly compressed in moving from a constricted dilation state to a dilated dilation state or in which the radial positions of the iris at each angular location are uniformly and linearly expanded in moving from a dilated dilation state to a constricted dilation state.

However, the iris structure generally does not expand or compress in a linear manner with respect to different radial positions. For example, the iris structure may expand and/or compress more towards the middle than at its edges. As such, the deformation function according to the present disclosure may use a different type of modeling in which the degree of expansion and/or compression of the iris structure may vary at different radial locations.

For example, according to one or more embodiments of the present disclosure, the deformation function may include a Gaussian function applied along the radial positions rather than a linear function. For instance, FIG. 3D illustrates an example plot 380 that illustrates relationships between radius positions of portions of the iris structure in resulting depiction modifications as compared to a reference depiction based on different distortion functions corresponding to compression modeling. In the plot 380, the x-axis represents the radial position of particular points of the iris structure along the same angular position in the modified depiction and the y-axis represents the radial position of the same points of the iris structure along the same angular position in the reference depiction. Further, the reference depiction corresponds to a distortion function that would be a strictly linear distortion function.

As indicated in the plot 380, the “Linear” line illustrates that in instances in which a strictly linear deformation function is used, the relationships between the distances of the different radial positions of points in the iris structure are the same for both the reference depiction and the modified depiction. For example in the reference image, radial positions r1 and r2 may have a distance “d12” between them. With a compression factor of 2 in a modified depiction with a linear deformation function, this distance may be halved—e.g., converted into d12 * 0.5. In the linear case, this holds for the interplay of all radial positions.

Further, as indicated in the plot 380, the “Linear+Gaussian” line illustrates that the differences between radial positions of points in the iris structure differ between the reference depiction and the modified depiction in a non-linear manner. For example, in a non-linear deformation function corresponding to a compression factor of 2, some relative distances between the radial positions of some points may have a factor of 0.5 applied to them and others may have a different factor (e.g., 0.4) applied to them, depending on the Gaussian function. Moreover, the “Gaussian” line of the plot 380 illustrates that the difference between the “Linear” line and the “Linear+Gaussian” line follows a Gaussian distribution.

The applying of the deformation function to the first depiction may result in generation of a first iteration of the modified depiction. In some embodiments, the block 304 may also include a block 316 corresponding to modifying the deformation function. In these or other embodiments, the modifying of the deformation function may be based on one or more iterations of the modified depiction.

For example, in some embodiments, the first iteration of the modified depiction of the first depiction may be compared against the second depiction of the eye in the second dilation state. The comparison may include comparing radial positions of same portions of the iris in the second depiction as compared to the first iteration of the modified depiction to identify differences between the radial positions. In these or other embodiments, one or more parameters of the deformation function may be adjusted based on such differences and the modified deformation function may be applied to the first depiction to obtain a new iteration of the modified depiction. In these or other embodiments, such a process may be repeated until the deformation function is such that the differences between the second depiction and the modified depiction are minimized and/or satisfy a certain threshold.

For example, in instances in which a Gaussian function is used, the amplitude, standard deviation, and/or center position of the Gaussian function may be modified and the modified Gaussian function may be applied to the first modified depiction to obtain a second modified depiction that may be compared against the second depiction. In these or other embodiments, one or more parameters of the Gaussian function may be modified further based on differences between the second modified depiction and the second depiction that may be indicated by the corresponding comparison. Additionally or alternatively, the process may be repeated multiple times until the Gaussian function is modified such that differences between the second depiction and the modified depiction are minimized and/or below a certain threshold.

In some embodiments, any suitable optimization technique or function may be used to adjust the deformation function. For example, in some instances, a loss function may be used to determine a loss that represents the differences and the process may be repeated until the loss is minimized and/or satisfies a certain threshold. In these or other embodiments, some example optimization techniques that may be used to reduce the loss may include the Nelder-Mead technique, a conjugate gradient technique, a Broyden–Fletcher–Goldfarb–Shanno or BFGS technique, or any other suitable technique.

In some embodiments, rather than or in addition to blocks 314 and/or 316 for the generation of modified depiction, the block 304 may include a block 318. At block 318 an AI model may be used to obtain the modified depiction of the first depiction. For example, in some embodiments the first image and the second image may be provided to the AI model. In these or other embodiments, the AI model may be configured to modify the first depiction based on the second depiction such that the degrees of radial adjustments to generate the modified depiction match or substantially match what would be applied to obtain the second depiction from the first depiction. As indicated above, the AI model may include any suitable AI model that may be configured (e.g., trained (e.g., using different images of same eyes at different dilation states)) to perform such operations.

In some embodiments, the process 300 may include a block 308. At block 308, the modified depiction that is obtained from the deformation function optimization and/or from the AI model use may be finalized into a third depiction of the eye that corresponds to the second dilation state. In the present disclosure, the modified depiction that may result from block 304 may also be referred to as “an optimized modified depiction,” but such a designation does not necessarily mean that such depiction is perfectly optimized. In some embodiments, the “finalization” of the modified depiction may merely include selecting and/or using the optimized modified depiction as the third depiction, which may be used for registration.

In these and other embodiments, the block 308 may include performing reverse pre-processing of the optimized modified depiction as part of finalizing the modified depiction into the third depiction. For example, the reverse pre-processing may include recovering the original image size of the cropped iris band in the modified depiction, reverting the shifting of each angle position that was previously done to align the radius of the pupil-iris boundary, and/or converting the modified depiction from the polar coordinate system into the cartesian coordinate system. The resulting depiction after the reverse pre-processing may be used as the third depiction.

The process 300 may accordingly be used to generate the third depiction based on the first and second depictions through the modeling of an ophthalmic structure (e.g., iris band). Modifications, additions, or omissions may be made to the process 300 without departing from the scope of the present disclosure. For example, two or more of the operations of process 300 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

In addition, as indicated with FIGS. 1 and 2, although the above description relates to generating the third depiction by modifying the first depiction and then performing the registration between the third depiction and the second depiction, other embodiments may include generating the third depiction by modifying the second depiction and then performing the registration between the third depiction and the first depiction. Moreover, although the examples of FIG. 3C relate to the first depiction 360a corresponding to the pupil being in a constricted state and the second depiction 360b corresponding to the pupil being in a dilated state, in other instances, the dilation states may be reversed. As such, in some instances the third depiction may correspond to a dilated state and in other instances the third depiction may correspond to a constricted or undilated state.

### EXAMPLE OPHTHALMIC SURGICAL SYSTEM

FIG. 4 is a block diagram of an example ophthalmic surgical system 400 (“system 400”) that may be used with and/or implement one or more embodiments of the present disclosure related to alignment registration of an eye. In general, the system 400 may be configured to perform one or more ophthalmic treatment operations with respect to a target tissue 412, which may be a cornea, a portion of a cornea, a lens, or a portion of a lens of an eye. In some embodiments, the system 400 may include a laser 402, an optics module 404, an imaging system 406, a control module 408, and a patient interface 410.

The laser 402 may include any suitable system, apparatus, or device, configured to generate one or more laser beams that may be used to perform ophthalmic operations or tasks with respect to the target tissue 412. In some embodiments, the laser 402 may include multiple lasers that each generate an individual laser beam. Additionally or alternatively, the laser 402 may include a single laser that is configured to generate a single beam or multiple beams.

In some embodiments, the laser 402 may be configured to generate a pulsed laser beam that is pulsed at a high repetition rate at a pulse repetition rate of thousands of shots per second or higher with relatively low energy per pulse. For example, in some embodiments, the laser 402 may be a femtosecond laser that emits ultra-short pulses of light (e.g., on the order of 10^⁻¹⁵ seconds). Such a laser may be operated to use relatively low energy per pulse to localize the tissue effect of the target tissue 412 that may be caused by laser-induced photodisruption by the beam generated by the laser 402.

The optics module 404 may include any suitable system, apparatus, or device that may be configured to focus and direct the laser beam to the target tissue 412. For example, in some embodiments, the optics module 404 may include one or more lenses and/or one or more reflectors (e.g., mirrors). Additionally or alternatively, in some embodiments, the optics module 404 may include one or more actuators that may be configured to adjust the focusing and/or the beam direction in response to a beam control signal that may be received from the control module 408. In these or other embodiments, the one or more actuators may be adjusted in response to a user input via any suitable user interface, such as discussed with respect to the computing system of FIG. 5. For example, in some embodiments, the user interface may include a touch screen, mouse, keyboard, joystick, foot pedal, game pad, game controller, etc., that may be used to provide commands for movement of laser beam (e.g., via the actuators).

The imaging system 406 may include any suitable system, apparatus, or device, that may be configured to obtain one or more images of the eye corresponding to the target tissue 412. For example, in some embodiments, the imaging system 406 may collect reflected or scattered light or sound from the target tissue 412 to capture image data corresponding to the target tissue 412.

The imaging system 406 may include one or more different types of devices and/or systems configured to capture various different types of images of the eye as corresponding to the target tissue 412. For example, in some embodiments, the imaging system 406 may include a camera configured to capture one or more camera images of the eye (e.g., such as those used to perform alignment registration as discussed in the present disclosure). Additionally or alternatively, the imaging system 406 may include an optical coherent tomography (OCT) device configured to capture OCT images of the eye. In these or other embodiments, the imaging system may include an ultrasound imaging device configured to capture ultrasound images of the eye.

The patient interface 410 may include a mount that is configured to engage with the target tissue 412 to hold the target tissue 412 in position during performance of the ophthalmic procedure. In these or other embodiments, the patient interface 410 may be configured to allow the laser beam to pass therethrough to allow for performance of the procedure via the laser beam.

The control module 408 may include any suitable system, apparatus, or device, configured to perform one or more control operations with respect to the system 400. For example, in some embodiments, the control module 408 may include code and routines configured to allow a computing system to perform one or more operations. Additionally or alternatively, the control module 408 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these or other embodiments, the control module 408 may be implemented using a combination of hardware and software. In the present disclosure, operations described as being performed by the control module 408 may include operations that the control module 408 may direct a corresponding computing system to perform. In these or other embodiments, the control module 408 may be implemented by one or more computing systems, such as that described in further detail with respect to FIG. 5 of the present disclosure.

In some embodiments, the control module 408 may be configured to control the laser 402 and/or the optics module 404. Additionally or alternatively, the control module 408 may be configured to control any number of other components of the system 400 not expressly illustrated.

Additionally or alternatively, the control module 408 may be configured to determine the placement of the laser beam and corresponding laser pulses with respect to the target tissue 412. For example, in some embodiments, the control module 408 may include a registration module such as described with respect to the registration module 102 of FIG. 1. The control module 408 may accordingly be configured in some embodiments to determine an alignment registration based on one or more images received from the imaging system 406 and/or one or more other diagnostic images that had been previously captured (e.g., during a diagnosis and via a different camera than that of the imaging system 406 and/or the same camera). In these or other embodiments, the control module 408 may be configured to determine the placement of the laser beam and the corresponding laser pulses based on the alignment registration.

Additionally or alternatively, the control module 408 may be configured to cause one or more adjustment operations (e.g., such as described in the present disclosure) to be performed based on the determined placement. For example, the control module 408 may control one or more actuators of the optics module 404 to adjust a location of the laser beam and corresponding laser pulses with respect to the target tissue 412. Additionally or alternatively, the control module 408 may adjust a pattern of the laser beam through adjustment of the laser 402 and/or the optics module 404 (e.g., to rotate a planned treatment according to the alignment registration). In these or other embodiments, the control module 408 may adjust one or more other actuators that may move the entire system 400 or the laser 402 itself such that the laser orientation may be moved with respect to the target tissue 412. Additionally or alternatively, the control module 408 may cause the adjustment of a support platform (e.g., bed) that the patient is lying on to adjust the orientation of the laser 402 with respect to the target tissue 412 (e.g., by controlling one or more actuators corresponding to the support platform).

Modifications, additions, or omissions may be made to FIG. 4 without departing from the scope of the present disclosure. For example, the system 400 may include more or fewer elements depending on the implementation. Further, the system 400 may be configured to perform any number of operations as compared to those explicitly described.

### EXAMPLE COMPUTING SYSTEM

FIG. 5 is a block diagram of an example computing system 500 suitable for use in implementing some embodiments of the present disclosure. Computing system 500 may include an interconnect system 502 that directly or indirectly couples the following devices: memory 504, one or more central processing units (CPUs) 506, one or more graphics processing units (GPUs) 508, a communication interface 510, I/O ports 512, input/output components 514, a power supply 516, one or more presentation components 518 (e.g., display(s)), and one or more logic units 520.

Although the various blocks of FIG. 5 are shown as connected via the interconnect system 502 with lines, this is not intended to be limiting and is for clarity only. For example, in some embodiments, a presentation component 518, such as a display device, may be considered an I/O component 514 (e.g., if the display is a touch screen). As another example, the CPUs 506 and/or GPUs 508 may include memory (e.g., the memory 504 may be representative of a storage device in addition to the memory of the GPUs 508, the CPUs 506, and/or other components). In other words, the computing system of FIG. 5 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 5.

The interconnect system 502 may represent one or more links or busses, such as an address bus, a data bus, a control bus, or a combination thereof. The interconnect system 502 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 506 may be directly connected to the memory 504. Further, the CPU 506 may be directly connected to the GPU 508. Where there is direct, or point-to-point, connection between components, the interconnect system 502 may include a PCIe link to carry out the connection. In these examples, a PCI bus need not be included in the computing system 500.

The memory 504 may include any of a variety of computer-readable media. The computer-readable media may be any available media that may be accessed by the computing system 500. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 504 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 500. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 506 may be configured to execute at least some of the computer-readable instructions to control one or more components of the computing system 500 to perform one or more of the methods and/or processes described herein. The CPU(s) 506 may each include one or more cores (e.g., one, two, four, eight, twenty-eight, seventy-two, etc.) that are capable of handling a multitude of software threads simultaneously. The CPU(s) 506 may include any type of processor and may include different types of processors depending on the type of computing system 500 implemented (e.g., processors with fewer cores for mobile devices and processors with more cores for servers). For example, depending on the type of computing system 500, the processor may be an Advanced RISC Machines (ARM) processor implemented using Reduced Instruction Set Computing (RISC) or an x86 processor implemented using Complex Instruction Set Computing (CISC). The computing system 500 may include one or more CPUs 506 in addition to one or more microprocessors or supplementary co-processors, such as math co-processors.

In addition to or alternatively from the CPU(s) 506, the GPU(s) 508 may be configured to execute at least some of the computer-readable instructions to control one or more components of the computing system 500 to perform one or more of the methods and/or processes described herein. One or more of the GPU(s) 508 may be an integrated GPU (e.g., with one or more of the CPU(s) 506 and/or one or more of the GPU(s) 508 may be a discrete GPU. In embodiments, one or more of the GPU(s) 508 may be a coprocessor of one or more of the CPU(s) 506. The GPU(s) 508 may be used by the computing system 500 to render graphics (e.g., 3D graphics) or perform general purpose computations. For example, the GPU(s) 508 may be used for General-Purpose computing on GPUs (GPGPU). The GPU(s) 508 may include hundreds or thousands of cores that are capable of handling hundreds or thousands of software threads simultaneously. The GPU(s) 508 may generate pixel data for output images in response to rendering commands (e.g., rendering commands from the CPU(s) 506 received via a host interface). The GPU(s) 508 may include graphics memory, such as display memory, for storing pixel data or any other suitable data, such as GPGPU data. The display memory may be included as part of the memory 504. The GPU(s) 508 may include two or more GPUs operating in parallel (e.g., via a link). The link may directly connect the GPUs or may connect the GPUs through a switch. When combined together, each GPU 508 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 506 and/or the GPU(s) 508, the logic unit(s) 520 may be configured to execute at least some of the computer-readable instructions to control one or more components of the computing system 500 to perform one or more of the methods and/or processes described herein. In embodiments, the CPU(s) 506, the GPU(s) 508, and/or the logic unit(s) 520 may discretely or jointly perform any combination of the methods, processes and/or portions thereof. One or more of the logic units 520 may be part of and/or integrated in one or more of the CPU(s) 506 and/or the GPU(s) 508 and/or one or more of the logic units 520 may be discrete components or otherwise external to the CPU(s) 506 and/or the GPU(s) 508. In embodiments, one or more of the logic units 520 may be a coprocessor of one or more of the CPU(s) 506 and/or one or more of the GPU(s) 508.

Examples of the logic unit(s) 520 include one or more processing cores and/or components thereof, such as Tensor Cores (TCs), Tensor Processing Units(TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 510 may include one or more receivers, transmitters, and/or transceivers that enable the computing system 500 to communicate with other computing systems via an electronic communication network, including wired and/or wireless communications. The communication interface 510 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 512 may enable the computing system 500 to be logically coupled to other devices including the I/O components 514, the presentation component(s) 518, and/or other components, some of which may be built into (e.g., integrated in) the computing system 500. Illustrative I/O components 514 include a microphone, mouse, keyboard, joystick, foot pedal, game pad, game controller, satellite dish, scanner, printer, wireless device, etc. The I/O components 514 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition associated with a display of the computing system 500. The computing system 500 may include depth cameras, such as stereoscopic camera systems, infrared camera systems, RGB camera systems, touchscreen technology, and combinations of these, for gesture detection and recognition. Additionally, the computing system 500 may include accelerometers or gyroscopes (e.g., as part of an inertia measurement unit (IMU)) that enable detection of motion. In some examples, the output of the accelerometers or gyroscopes may be used by the computing system 500 to render immersive augmented reality or virtual reality.

The power supply 516 may include a hard-wired power supply, a battery power supply, or a combination thereof. The power supply 516 may provide power to the computing system 500 to enable the components of the computing system 500 to operate.

The presentation component(s) 518 may include a display (e.g., a monitor, a touch screen, a television screen, a heads-up-display (HUD), other display types, or a combination thereof), speakers, and/or other presentation components. The presentation component(s) 518 may receive data from other components (e.g., the GPU(s) 508, the CPU(s) 506, etc.), and output the data (e.g., as an image, video, sound, etc.).

Modifications, additions, or omissions may be made to FIG. 5 without departing from the scope of the present disclosure. For example, the system 500 may include more or fewer elements depending on the implementation. Further, the system 500 may be configured to perform any number of operations as compared to those explicitly described.

The present disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 3, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 3) or examples, or combined with aspects of another example The following is a non-limiting summary of some example implementations presented herein.

Example 1. A system comprising:

a computing system configured to cause performance of operations, the operations comprising:

obtaining a first image of a first depiction of an eye in a first dilation state;

obtaining a second image of a second depiction of the eye in a second dilation state;

generating a third depiction of the eye in the second dilation state, the generating of the third depiction including modifying the first depiction based on the second depiction; and

determining an alignment registration between the first depiction of the eye and the second depiction of the eye based on a comparison between the second depiction and the third depiction.

Example 2. The system of Example 1, wherein the generating of the third depiction includes:

applying a deformation function to the first depiction to obtain a modified depiction of the eye as compared to the first depiction, the applying of the deformation function including adjusting the first dilation state of the eye at respective radial positions of an iris in a non-linear manner based on a difference between the first dilation state and the second dilation state; and

generating the third depiction of the eye based on the modified depiction of the eye.

Example 3. The system of any of Example 1 or Example 2, wherein the generating of the third depiction includes:

modifying the deformation function based on a comparison between the modified depiction and the second depiction;

adjusting the modified depiction of the eye based on the deformation function as modified; and

generating the third depiction of the eye based on the modified depiction as adjusted.

Example 4. The system of Example 3, wherein the modifying of the deformation function includes modifying one or more parameters of the deformation function based on an optimization function.

Example 5. The system of any of Examples 2-4, wherein the deformation function includes a combination of a linear function and a Gaussian function.

Example 6. The system of any of Examples 1-5, wherein the modifying of the first depiction based on the second depiction is such that a first structural state of an iris of the eye in the first depiction is modified in a manner in which a third structural state of the iris in the third depiction matches a second structural state of the iris in the second depiction.

Example 7. The system of any of Examples 1, 2, or 6, wherein the generating of the third depiction includes providing the first image and the second image to an artificial intelligence model that is trained to modify the first depiction to match the second dilation state of the second depiction to obtain the third depiction.

Example 8. The system of any of Examples 1-7, wherein the determining of the alignment registration is based on alignment in the second depiction and the third depiction of one or more of a first transition edge of the eye or a second transition edge of the eye.

Example 9. The system of Example 8, wherein:

the first transition edge corresponds to a border between a pupil and an iris of the eye; and

the second transition edge corresponds to a border between the iris and a sclera of the eye.

Example 10. The system of any of Examples 1-9, wherein the determining of the alignment registration includes determining a cyclotorsion angle between the first depiction and the second depiction.

Example 11. The system of any of Examples 1-10, further comprising a laser configured to emit a laser beam used to perform one or more ophthalmic treatment operations, the laser beam being adjusted based on the alignment registration.

Example 12. The system of Example 11, wherein the adjustment of the laser beam includes adjusting an orientation of the laser beam with respect to the eye.

Example 13. The system of Example 12, wherein adjusting the orientation of the laser beam with respect to the eye includes one or more of:

adjusting a position of the laser beam with respect to the eye; or

adjusting a pattern of the laser beam.

Example 14. The system of Example 13, further comprising one or more actuators configured to adjust the position of the laser beam.

Example 15. The system of Example 14, wherein the one or more actuators are configured to adjust the position of the laser beam via one or more of:

adjusting one or more reflectors that reflect at least a portion of the laser beam;

adjusting an overall position of the laser; or

adjusting a position of a support on which a patient corresponding to the eye is laying.

Example 16. The system of any of Examples 11-15, further comprising a patient interface configured to interface with the eye and configured to have the laser beam pass therethrough.

Example 17. The system of any of Examples 1-16, wherein the first image is captured using a first camera separate from the system.

Example 18. The system of Example 17, further comprising a second camera configured to capture the second image.

Example 19. The system of any of Examples 1-18, wherein the first dilation state is a dilated state and the second dilation state is a constricted state.

Example 20. The system of any of Examples 1-19, wherein the first dilation state is a constricted state and the second dilation state is a dilated state.

Example 21. A method comprising:

generating, based on a first image of a first depiction of an eye in a first dilation state and a second image of a second depiction of the eye in a second dilation state, a third depiction of the eye in the second dilation state; and

determining an alignment registration between the first depiction of the eye and the second depiction of the eye based on a comparison between the second depiction and the third depiction.

Example 22. The method of Example 21, wherein the generating of the third depiction includes modifying the first depiction based on the second depiction such that a first structural state of an iris of the eye in the first depiction is modified in a manner in which a third structural state of the iris in the third depiction matches a second structural state of the iris in the second depiction.

Example23. The method of any of Example 21 or Example 22, wherein the generating of the third depiction includes:

applying a non-linear deformation function to the first depiction to obtain a modified depiction of the eye as compared to the first depiction;

modifying the deformation function based on a comparison between the modified depiction and the second depiction;

adjusting the modified depiction of the eye based on the deformation function as modified; and

generating the third depiction of the eye based on the modified depiction as adjusted.

Example 24. The method any of Examples 21-23, wherein the deformation function includes a combination of a linear function and a Gaussian function.

Example 25. The method of any of Example 21 or Example 22, wherein the generating of the third depiction includes providing the first image and the second image to an artificial intelligence model that is trained to modify the first depiction to match the second dilation state of the second depiction to obtain the third depiction.

What is claimed is:

1. A system comprising:
a computing system configured to cause performance of operations, the operations comprising:
obtaining a first image of a first depiction of an eye in a first dilation state;
obtaining a second image of a second depiction of the eye in a second dilation state;
generating a third depiction of the eye in the second dilation state, the generating of the third depiction including modifying the first depiction based on the second depiction; and
determining an alignment registration between the first depiction of the eye and the second depiction of the eye based on a comparison between the second depiction and the third depiction.

2. The system of claim 1, wherein the generating of the third depiction includes:
applying a deformation function to the first depiction to obtain a modified depiction of the eye as compared to the first depiction, the applying of the deformation function including adjusting the first dilation state of the eye at respective radial positions of an iris in a non-linear manner based on a difference between the first dilation state and the second dilation state; and
generating the third depiction of the eye based on the modified depiction of the eye.

3. The system of claim 1, wherein the generating of the third depiction includes:
modifying the deformation function based on a comparison between the modified depiction and the second depiction;
adjusting the modified depiction of the eye based on the deformation function as modified; and
generating the third depiction of the eye based on the modified depiction as adjusted.

4. The system of claim 3, wherein the modifying of the deformation function includes modifying one or more parameters of the deformation function based on an optimization function.

5. The system of claim 2, wherein the deformation function includes a combination of a linear function and a Gaussian function.

6. The system of claim 1, wherein the modifying of the first depiction based on the second depiction is such that a first structural state of an iris of the eye in the first depiction is modified in a manner in which a third structural state of the iris in the third depiction matches a second structural state of the iris in the second depiction.

7. The system of claim 1, wherein the generating of the third depiction includes providing the first image and the second image to an artificial intelligence model that is trained to modify the first depiction to match the second dilation state of the second depiction to obtain the third depiction.

8. The system of claim 1, wherein the determining of the alignment registration is based on alignment in the second depiction and the third depiction of one or more of a first transition edge of the eye or a second transition edge of the eye.

9. The system of claim 8, wherein:
the first transition edge corresponds to a border between a pupil and an iris of the eye; and
the second transition edge corresponds to a border between the iris and a sclera of the eye.

10. The system of claim 1, wherein the determining of the alignment registration includes determining a cyclotorsion angle between the first depiction and the second depiction.

11. The system of claim 1, further comprising a laser configured to emit a laser beam used to perform one or more ophthalmic treatment operations, the laser beam being adjusted based on the alignment registration.

12. The system of claim 1, further comprising one or more actuators configured to adjust a position of the laser beam.

13. The system of claim 11, further comprising a patient interface configured to interface with the eye and configured to have the laser beam pass therethrough.

14. The system of claim 1, wherein the first image is captured using a first camera separate from the system and wherein the system further comprises a second camera configured to capture the second image.

15. The system of claim 1, wherein the first dilation state is a dilated state and the second dilation state is a constricted state.

16. The system of claim 1, wherein the first dilation state is a constricted state and the second dilation state is a dilated state.

17. A method comprising:
generating, based on a first image of a first depiction of an eye in a first dilation state and a second image of a second depiction of the eye in a second dilation state, a third depiction of the eye in the second dilation state; and
determining an alignment registration between the first depiction of the eye and the second depiction of the eye based on a comparison between the second depiction and the third depiction.

18. The method of claim 17, wherein the generating of the third depiction includes modifying the first depiction based on the second depiction such that a first structural state of an iris of the eye in the first depiction is modified in a manner in which a third structural state of the iris in the third depiction matches a second structural state of the iris in the second depiction.

19. The method of claim 17, wherein the generating of the third depiction includes:
applying a non-linear deformation function to the first depiction to obtain a modified depiction of the eye as compared to the first depiction;
modifying the deformation function based on a comparison between the modified depiction and the second depiction;
adjusting the modified depiction of the eye based on the deformation function as modified; and
generating the third depiction of the eye based on the modified depiction as adjusted.

20. The method of claim 17, wherein the generating of the third depiction includes providing the first image and the second image to an artificial intelligence model that is trained to modify the first depiction to match the second dilation state of the second depiction to obtain the third depiction.
